(19) United States
(12) Patent Application Publication
(10) Pub. No.: US 2013/0309659 A1
(43) Pub. Date: Nov. 21, 2013

(54) CELL CULTURING FORMULATION AND CULTURING AND QUANTIFICATION METHOD OF CD140B+ CELLS THEREOF

(71) Applicant: CHAO-HUNG WANG, Taoyuan County (TW)

(72) Inventor: CHAO-HUNG WANG, Taoyuan County (TW)

(73) Assignee: CHANG-GUNG UNIVERSITY, Taoyuan County (TW)

(21) Appl. No.: 13/614,044

(22) Filed: Sep. 13, 2012

(30) Foreign Application Priority Data
May 16, 2012 (TW) 101117485

(51) Int. Cl. C12N 5/071; C12Q 1/68; C12Q 1/25; C12Q 1/02

(52) U.S. Cl. 435/6.1; 435/408; 435/405; 435/29

(57) ABSTRACT

The present invention discloses a cell culturing formulation and a culturing and quantification method of CD140b+ cells thereof. The cell culturing formulation is applicable for inducing the growth of the CD140b+ cells in peripheral blood. The cell culturing formulation comprises a culturing medium, a serum, a mixed additive and a defined factor. Wherein, concentrations of the culturing medium, the serum, the mixed additive and the defined factor are 59˜98% (v/v), 0.1˜20% (v/v), 1˜10% (v/v) and 10⁻⁷˜10% (v/v) respectively.

### CROSS-REFERENCE TO RELATED APPLICATION

This application claims the benefit under 35 U.S.C. §119 of Taiwanese Patent Application No. 101117485, filed May 16, 2012, which is hereby incorporated by reference in its entirety.

### BACKGROUND

1. Field of the Invention

The present invention relates to a cell culturing formulation and a culturing and quantification method of CD140b+ cells, and more particularly to the cell culturing formulation and the culturing and quantification method of CD140b+ cells capable of effectively culturing CD140b+ cells with a better CD140b expression by using a new formulation of cell culturing medium.

2. Description of Related Art

Arteriosclerosis is an inflammatory disease of artery and is a vascular disease that occurs more frequently with aging. It generally occurs at young age and becomes more serious or outbreak more frequently at middle to old age, and there are more male patients than female patients of vascular diseases. In recent years, increasingly more arteriosclerosis patients are found in Taiwan, and arteriosclerosis has become one of the major causes of death of the elderly. In developed countries, arteriosclerosis is the most common cause of death and is always an important subject for medical and biological research.

In general, arteriosclerosis is caused by chronic degeneration and gradual changes of arterial walls. In particularly, arteriosclerosis is caused by abnormal elongation and aggregation of connective tissues (such as smooth muscle cells or smooth muscle progenitor cells) in vessel walls, such that the arterial walls becomes thicker and harder and the artery diameter becomes smaller, and finally the whole artery loses its elasticity and becomes obstructed. Clinically, quantification of smooth muscle progenitor cell (SMPC) (CD140b+ cells) in blood circulation can be applied for examining cardiovascular diseases, assessing the effect of cardiovascular disease treatments, evaluating cardiovascular disease risks, and performing prognostic evaluations of cardiovascular diseases. Therefore, the amount of smooth muscle progenitor cells (SMPC) (CD140b+ cells) in blood circulation is very important to the risk evaluation of the arteriosclerosis. In addition, quantification of smooth muscle progenitor cell (SMPC) (CD140b+ cells) in blood circulation can also be applied to cancer evaluation and prognostic reference as well as wound healing evaluation and prognostic reference.

However, the quantity of CD140b+ cells in blood is very little and the cells cannot be cultured easily. When a flow cytometry is used for counting the cells, the accuracy of the counting is always questionable and the challenge of the difficulty in counting is relatively high due to insufficient CD140b antigen expressions on the cell surface. In addition, conventional culturing media used for culturing CD140b+ cells in blood are expensive, and the quantity of the cultured CD140b+ cells is small, and the culturing time is long, so that the quantification of CD140b+ cells is non-scientific, instable, and has low experiment-repeatability.

Based on the aforementioned problems, the present invention provides a new formulation of a cell culturing medium to culture CD140b+ cells with a better CD140b expression.

### BRIEF SUMMARY

In view of the aforementioned problems of the prior art, it is a primary objective of the invention to provide a cell culturing formulation and a culturing and quantification method of CD140b+ cells thereof to overcome the problems of the conventional cell culturing medium that fails to culture CD140b+ cells easily, takes too much culturing time, incurring low stability, and having unrepeatable experiments.

To achieve the foregoing objective, the present invention provides a cell culturing formulation applicable for inducing the differentiation and growth of CD140b+ cells in peripheral blood to achieve a more accurate and simpler quantification, and the cell culturing formulation comprises a culturing medium, a serum, a mixed additive and a defined factor, wherein concentrations of the culturing medium, the serum, the mixed additive and the defined factor are 59˜98% (v/v), 0.1˜20% (v/v), 1˜10% (v/v) and 10⁻⁷˜10% (v/v) respectively.

Preferably, the serum includes a fetal bovine serum (FBS), a newborn calf serum (NCS) or a calf serum (CS).

Preferably, the defined factor includes a growth factor, a chemokine, or a cytokine.

Preferably, the growth factor includes a transforming growth factor beta (TGF-β) or a calcitonin gene related peptide (CGRP).

Preferably, the culturing medium includes a Dulbecco's modified eagle medium (DMEM), a Roswell Park Memorial Institute (RPMI) medium or an essential culturing medium.

Preferably, the CD140b+ cell includes a smooth muscle progenitor cell (SMPC), a circulating fibrocyte or a cancer cell.

Preferably, the mixed additive includes L-glutamine, minimum essential medium non-essential amino acid (MEM NEAA) solution, sodium pyruvate or penicillin/streptomycin (Pen/Strep).

To achieve the foregoing objective, the present invention further provides a culturing and quantification method of CD140b+ cells, comprising the steps of: collecting a mononuclear cell from blood by using a centrifuge; mixing the mononuclear cell with a cell culturing formulation uniformly to form a mixed cell solution; and placing the mixed cell solution in a culture dish, and then placing the culture dish in a cell culture incubator to perform a culture for a predetermined time to obtain the CD140b+ cell. Wherein, the cell culturing formulation comprises a culturing medium, a serum, a mixed additive and a defined factor, and concentration of the culturing medium, the serum, the mixed additive and the defined factor are 59˜98% (v/v), 0.1˜20% (v/v), 1˜10% (v/v) and 10⁻⁷˜10% (v/v) respectively.

Preferably, the predetermined time falls within a range of 1˜7 days.

Preferably, the mixed additive includes L-glutamine, minimum essential medium non-essential amino acid (MEM NEAA) solution, sodium pyruvate or penicillin/streptomycin (Pen/Strep).

Preferably, the CD140b+ cell includes a smooth muscle progenitor cell (SMPC), a circulating fibrocyte or a cancer cell.

Preferably, the culturing and quantification method of CD140b+ cells includes a flow cytometry, a RNA quantification or an enzyme quantification method.

In summation, the cell culturing formulation and the culturing and quantification method of CD140b+ cells in accordance with the present invention adopt a new formulation of a cell culturing medium to culture CD140b+ cells, so that the CD140b+ cells can be grown in a large quantity rapidly, and a CD140b antigen with better expression can be achieved to overcome the problems of the conventional culturing media with having an expensive price, and the difficulty of culturing and quantifying CD140b+ cells.

### BRIEF DESCRIPTION OF THE DRAWINGS

FIG. 1 is a flow chart of a culturing method of CD140b+ cells in accordance with the present invention;

FIG. 2 is a schematic view of flow cytometry analyses showing the changes of cells from Day 0 to Day 3 in a process of culturing CD140b+ cells by a cell culturing formulation of the present invention;

FIG. 3 is a schematic view of flow cytometry analyses showing a process of marking CD140b and VE-cadherin positive cells cultured by a cell culturing formulation of the present invention;

FIGS. 4A to 4C are schematic views of flow cytometry analyses showing the cell populations of cells cultured by a TGF-β cell culturing formulation of the present invention;

FIGS. 5A to 5F are schematic views of analyses of animal experiments on CD140b+ cells cultured in accordance with the present invention.

### DETAILED DESCRIPTION

The technical contents and characteristics of the present invention will be apparent with the detailed description of a preferred embodiment accompanied with related drawings as follows. For simplicity, same numerals are used in the following preferred embodiment to represent respective same elements.

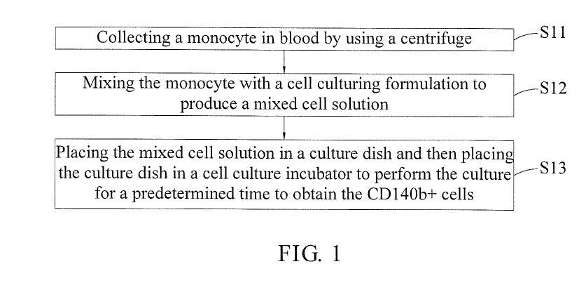

With reference to FIG. 1 for a flow chart of a culturing method of CD140b+ cells in accordance with the present invention, the culturing method of CD140b+ cells comprises the following steps:

S11: Collecting a mononuclear cell in blood by using a centrifuge.

S12: Mixing the mononuclear cell with a cell culturing formulation to produce a mixed cell solution.

S13: Placing the mixed cell solution in a culture dish, and then placing the culture dish in a cell culture incubator to perform the culture for a predetermined time to obtain the CD140b+ cells.

Wherein, the cell culturing formulation comprises a culturing medium, a serum, a mixed additive and a defined factor, and concentration of the culturing medium, the serum, the mixed additive and the defined factor are 59˜98% (v/v), 0.1˜20% (v/v), 1˜10% (v/v) and 10⁻⁷˜10% (v/v) respectively, and the concentration of the defined factor is preferably 0.01 pg˜100 mg/ml. The culturing medium includes a Dulbecco's Modified eagle medium (DMEM), a Roswell Park Memorial Institute (RPMI) medium or an essential culturing medium. The serum includes a fetal bovine serum (FBS), a newborn calf serum (NCS) or a calf serum (CS). The defined factor includes a growth factor, a chemokine or a cytokine, and the growth factor includes a transforming growth factor beta (TGF-β) or a calcitonin gene related peptide (CGRP). Further, the mixed additive includes L-glutamine, minimum essential medium non-essential amino acid (MEM NEAA) solution, sodium pyruvate or penicillin/streptomycin (Pen/Strep). Wherein the CD140b+ cell is a smooth muscle progenitor cell (SMPC), a circulating fibrocyte or a cancer cell. However, these embodiments are provided for the purpose of illustrating the present invention, but not intended for limiting the scope of the invention.

Wherein, the predetermined time for the cell culture is 1˜7 days. The diameter of the culture dish is equal to 10 cm. The temperature of the cell culture incubator is set to 37° C. These conditions are provided as examples, but the invention is not limited to these conditions only.

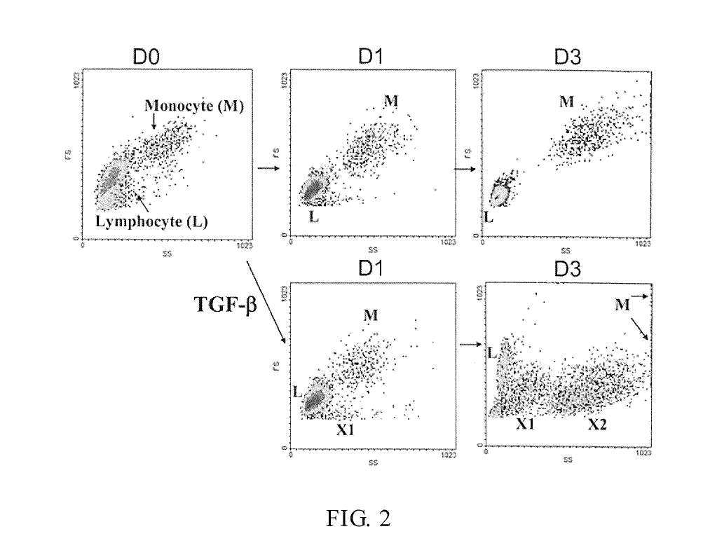

With reference to FIG. 2 for a schematic view of flow cytometry analyses showing the changes of cells from Day 0 to Day 3 in a process of culturing CD140b+ cells by a cell culturing formulation of the present invention, the collected blood is centrifuged to collect mononuclear cells in the blood, and then the mononuclear cells are suspended in the cell culturing formulation of the present invention and placed into a cell culture incubator containing 5% carbon dioxide and having a saturated humid environment at 37° C., and a cell analysis is preformed. The change of cells from Day 0 to Day 3 is analyzed. The result shows that if no TGF-β is added into the culturing medium, then the cultured cells only show two kinds of cell populations, respectively: lymphocyte (L) and monocyte (M) after Day 3. On the other hand, if TGF-β is added into the culturing medium, the cultured cells are divided into four cell populations, respectively: lymphocyte (L), monocyte (M) and X1 and X2 populations after Day 3.

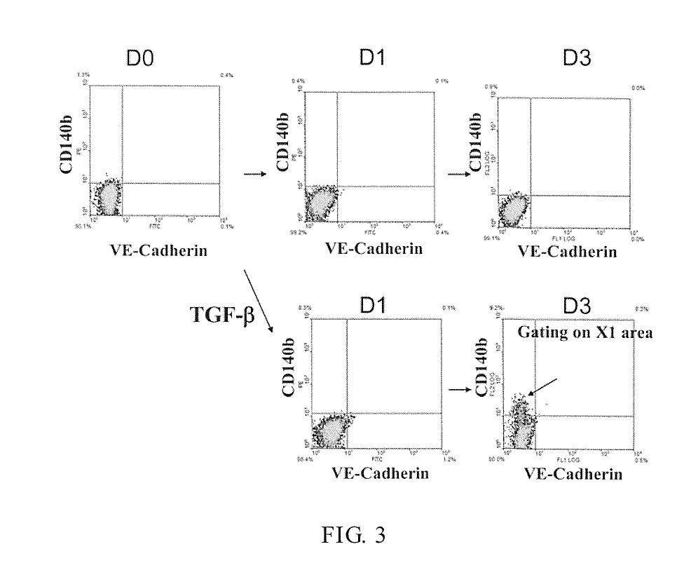

With reference to FIG. 3 for a schematic view of flow cytometry analyses showing a process of marking CD140b and VE-cadherin positive cells cultured by a cell culturing formulation of the present invention, the condition for the cell culture is mainly divided into two parts: (1) without adding any transforming growth factor beta (TGF-β) and (2) adding a transforming growth factor beta (TGF-β). After cells are cultured, the cells cultured by different culture conditions after Day 1 and Day 3 are marked by CD140b antigen, and the specific immune binding response between antigen and antibody is used to perform the analysis test in order to analyze the change of cells from Day 0 to Day 3. Observations show that if TGF-β is not added into the culturing medium, no CD140b surface antigen can be marked on the cultured cells or only a weak CD140b expression can be marked on the cultured cells after Day 0 to Day 3. Thus, results show that none or very little positive cells with a CD140b expression can be found in this culture condition. On the other hand, if TGF-β is added into the culturing medium, a large quantity of the cultured cells with a high expression of CD140 surface antigen can be detected in day 3, indicating that the cell culturing formulation with TGF-β can improve the CD 140b expression of CD140b+ cells significantly to improve the computing accuracy, and the CD140b positive cells have VE-cadherin negative responses. Wherein, VE-cadherin is an endothelial cell marker, and CD140b is a smooth muscle progenitor cell (SMPC) marker.

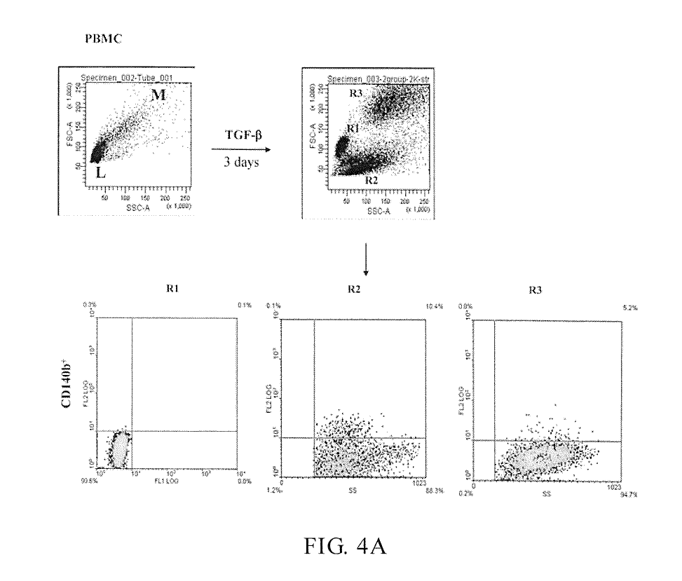

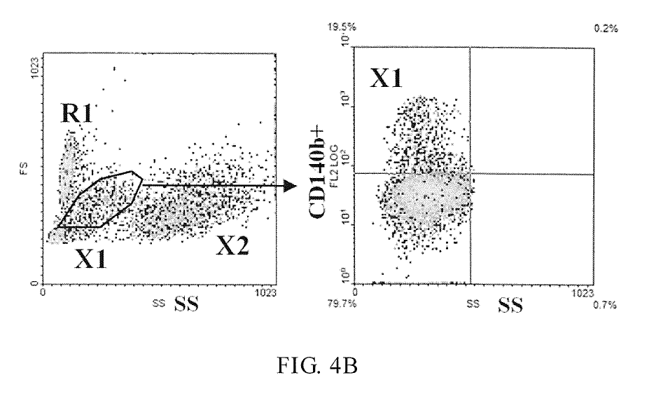

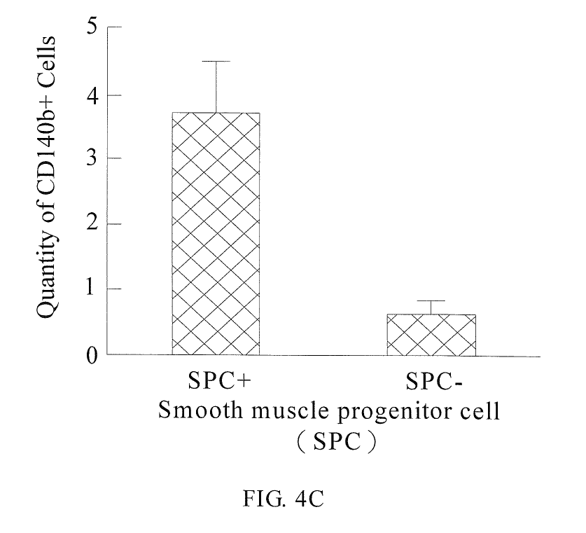

With reference to FIGS. 4A to 4C for schematic views of flow cytometry analyses showing the cell populations of cells cultured by a TGF-β cell culturing formulation of the present invention, blood is centrifuged and purified, and then a flow cytometry of the cell precipitates is performed to obtain two cell populations: lymphocyte (L) and monocyte (M). If the cell culturing formulation with transforming growth factor beta (TGF-β) is used for the cell culture, results show that the cells cultured by the TGF-β induce differentiation into R1, R2 and R3 cell populations after Day 3, and the CD140b positive cells fall in the R2 cell population as shown in FIG. 4A. If the R2 cell population is analyzed further, the R2 cell population can be divided into X1 and X2 cell populations, and CD140b positive cells with CD140b surface antigen falls into the X1 cell population as shown in FIG. 4B. If the blood extracted from a patient is separated, the cell culturing formulation of the present invention is used for cell culture for three days, more CD140b positive cells can be detected in the patient, and we have a better chance of culturing mature smooth muscle progenitor cells (SMPCs) as shown in FIG. 4C. The aforementioned results indicate that such short-term cell culturing formulation and quantification method can predict a higher successful rate of culturing out smooth muscle progenitor cells (SMPCs) from peripheral blood effectively.

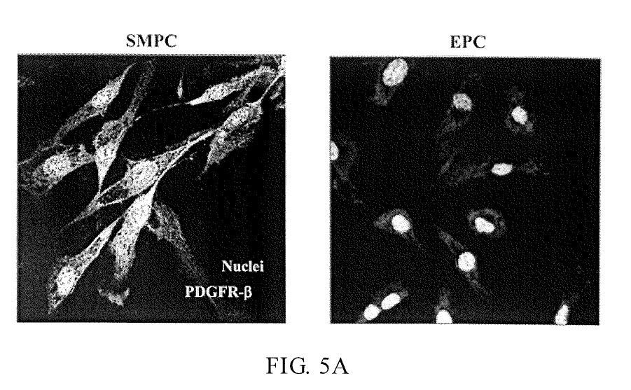

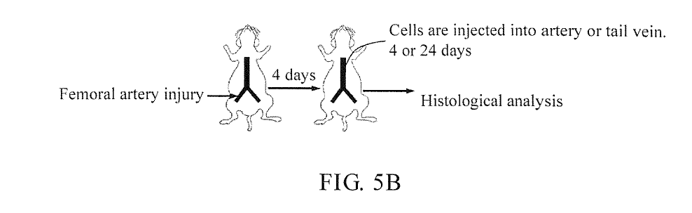

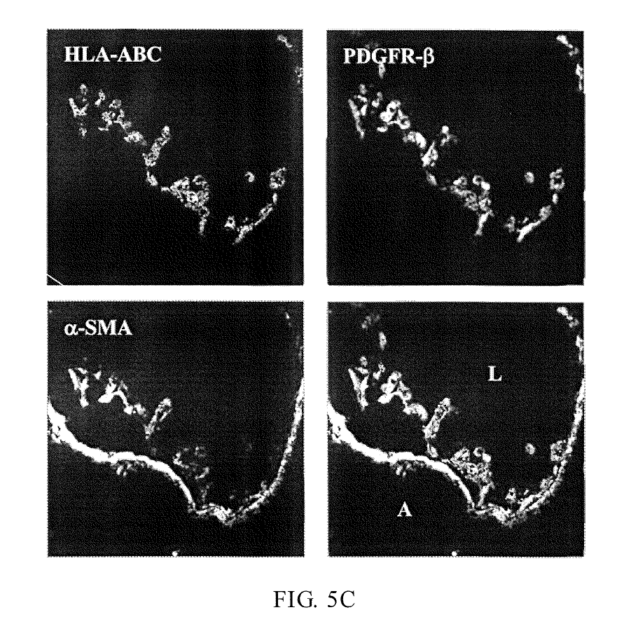

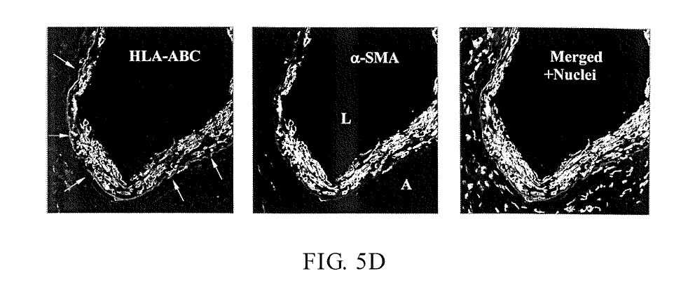

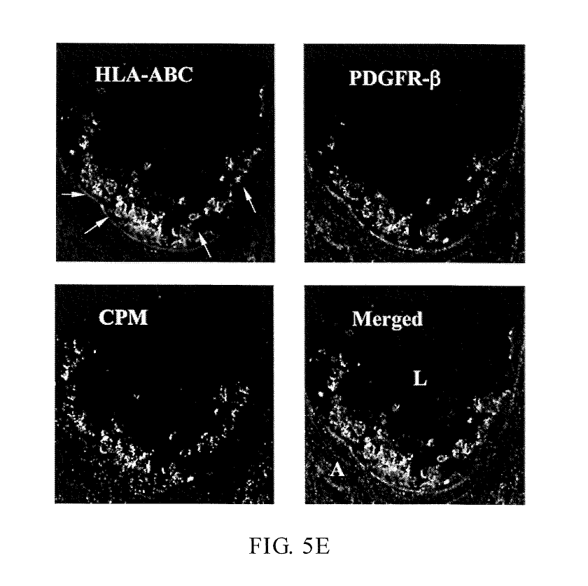

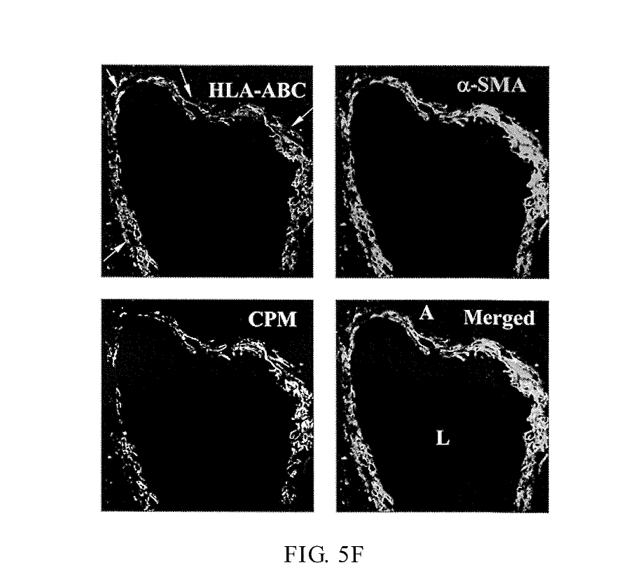

With reference to FIGS. 5A to 5F for schematic views of analyses of animal experiments on CD140b+ cells cultured in accordance with the present invention, results show that the smooth muscle progenitor cells (SMPC) cultured from peripheral blood can show PDGF-β receptor (CD140b), but the endothelial progenitor cells (EPCs) do not show CD140b as shown in FIG. 5A. The analysis of the animal experiment is described as follows. A spiral wire is used to cause an injury of the femoral artery of a mouse's thigh, and then after four days, the mature cultured smooth muscle progenitor cells (SMPC) (CD140b+ cells) or the purified mononuclear cells from peripheral blood are injected into the mouse. After 4 or 24 days, an immunostaining analysis of the mouse's injured blood vessel is performed as shown in FIG. 5B. After 4 days, immunostaining of the mouse's injured blood vessel with injected mature smooth muscle progenitor cells (SMPCs) is performed, and the results clearly show that the human smooth muscle progenitor cells (SMPCs) on the mouse's injured blood vessel show the properties of the smooth muscle progenitor cells (SMPCs) with both CD140b and α-SMA (smooth muscle actin) positive at the same time as shown in FIG. 5C. At 24 days after cells which are mononuclear cells purified from peripheral blood are injected, the cells will attach on the injured blood vessel wall and differentiate into α-SMA positive cells, showing that human smooth muscle progenitor cells (SMPCs) exist in the mononuclear cell population and can differentiate into smooth muscle progenitor cells (SMPC) as shown in FIG. 5D. In early vascular pathological analyses, we found that the injected human mononuclear cells are attached onto the mouse's injured blood vessel wall 4 days after the injection, and the mononuclear cells are CD140b positive cells as shown in FIG. 5E. Further, if mononuclear cells of peripheral blood are injected, the injected CD140b positive mononuclear cells differentiate into α-SMA positive smooth muscle progenitor cells (SMPCs) 24 days after the injection as shown in FIG. 5F. Wherein, the arrow in the figure indicates the elastic laminae in blood vessel, and HLA-ABC indicates that the cells come from human beings (L stands for blood vessel lumen and A stands for vascular peripheral tissues). Therefore, these experiments show that the cells quantified by the cell culturing formulation of the present invention can differentiate into mature smooth muscle progenitor cells (SMPCs) in animals.

In summation of the description above, the cell culturing formulation and the culturing and quantification method of CD140b+ cell in accordance with the present invention adopt a new formulation of the cell culturing medium to culture CD140b+ cells, such that the CD140b expression can be improved significantly, and the CD140b+ cells cultured by this cell culturing formulation have the features of a high stability of cell counting and easy replication of experiments.

1. A cell culturing formulation, applicable for inducing a growth of CD 140b+ cells in peripheral blood, comprising:
a culturing medium;
a serum;
a mixed additive; and
a defined factor;
wherein, concentrations of the culturing medium, the serum, the mixed additive and the defined factor are 59˜98% (v/v), 0.1˜20% (v/v), 1˜10% (v/v) and 10⁻⁷˜10% (v/v) respectively.

2. The cell culturing formulation of claim 1, wherein the serum includes a fetal bovine serum (FBS), a newborn calf serum (NCS) or a calf serum (CS).

3. The cell culturing formulation of claim 1, wherein the defined factor includes a growth factor, a chemokine or a cytokine.

4. The cell culturing formulation of claim 3, wherein the growth factor includes a transforming growth factor beta (TGF-β) or a calcitonin gene related peptide (CGRP).

5. The cell culturing formulation of claim 1, wherein the culturing medium includes a Dulbecco's modified eagle medium (DMEM), a Roswell Park Memorial Institute (RPMI) medium or an essential culturing medium.

6. The cell culturing formulation of claim 1, wherein the CD140b+ cell includes a smooth muscle progenitor cell (SMPC), a circulating fibrocyte or a cancer cell.

7. The cell culturing formulation of claim 1, wherein the mixed additive includes L-glutamine, minimum essential medium non-essential amino acid (MEM NEAA) solution, sodium pyruvate or penicillin/streptomycin(Pen/Strep).

8. A culturing and quantification method of CD140b+ cells, comprising the steps of:
collecting a mononuclear cell from blood by using a centrifuge;
mixing the mononuclear cell with a cell culturing formulation uniformly to form a mixed cell solution; and
placing the mixed cell solution in a culture dish, and then placing the culture dish in a cell culture incubator to perform a culture for a predetermined time to obtain the CD140b+ cell;
wherein, the cell culturing formulation comprises a culturing medium, a serum, a mixed additive and a defined factor, and concentration of the culturing medium, the serum, the mixed additive and the defined factor are 59˜98% (v/v), 0.1˜20% (v/v), 1˜10% (v/v) and 10⁻⁷˜10% (v/v) respectively.

9. The culturing and quantification method of CD 140b+ cells of claim 8, wherein the predetermined time falls within a range from 1 to 7 days.

10. The culturing and quantification method of CD140b+ cells of claim 8, wherein the mixed additive includes L-glutamine, minimum essential medium non-essential amino acid (MEM NEAA) solution, sodium pyruvate and penicillin/streptomycin (Pen/Strep).

11. The culturing and quantification method of CD140b+ cells of claim 8, wherein the CD140b+ cell includes a smooth muscle progenitor cell (SMPC), a circulating fibrocyte or a cancer cell.

12. The culturing and quantification method of CD140b+ cells of claim 8, wherein the CD140b+ cells are quantified by a flow cytometry, a RNA quantification or an enzyme quantification method.

13. The cell culturing formulation of claim 8, wherein the serum includes a fetal bovine serum (FBS), a newborn calf serum (NCS) or a calf serum (CS).

14. The cell culturing formulation of claim 8, wherein the defined factor includes a growth factor, a chemokine or a cytokine.

15. The cell culturing formulation of claim 14, wherein the growth factor includes a transforming growth factor beta (TGF-β) or a calcitonin gene related peptide (CGRP).

16. The cell culturing formulation of claim 8, wherein the culturing medium includes a Dulbecco's modified eagle medium (DMEM), a Roswell Park Memorial Institute (RPMI) medium or an essential culturing medium.
